(19) United States
(12) Patent Application Publication
(10) Pub. No.: US 2006/0222560 A1
(43) Pub. Date: Oct. 5, 2006

(54) Stuffed toy air-fragrance dispensing device

(76) Inventor: Virgil E. Stanley III, Indianapolis, IN (US)

Correspondence Address: VIRGIL E STANLEY;PATTY XIDIAS, 5860 N MICHIGAN ROAD, INDIANAPOLIS, IN, 46228, US

(21) Appl. No.: 11/377,659

(22) Filed: Mar. 9, 2006

Related U.S. Application Data
(63) Continuation-in-part of application No. 10/797,490, filed on Mar. 10, 2004.
(63) Continuation-in-part of application No. 10/164,818, filed on Jun. 7, 2002, now Pat. No. 6,830,733.
(63) Continuation-in-part of application No. 10/756,224, filed on Jan. 13, 2004.
(63) Continuation-in-part of application No. 10/959,267, filed on Oct. 6, 2004.

(51) Int. Cl. A61L 9/02

(52) U.S. Cl. 422/5; 422/124; 422/125

(57) ABSTRACT

A fragrance source is associated with the stuffed toy. In one embodiment, the fragrance source is disposed within a container contained within the stuffed toy or disposed within a pouch adjacent the stuffed toy. Air is directed past the fragrance source where the fragrance is mixed with the air to form an air-fragrance mixture. This air-fragrance mixture is directed upwardly through the hollow stem of the stuffed toy or its ornamental object and eventually emitted from the stuffed toy. Preferably the fragrance would be selected such that the scent emitted would simulate a scent which compliments the surrounding environment and its occupants.

### CROSS REFERENCE TO RELATED APPLICATIONS

The present application is a continuation-in-part of U.S. patent application Ser. No. 10/959,267 filed Oct. 6, 2004 entitled “Artificial Flower with Electric Fan and a Fragrance Source;” which is a continuation-in-part of U.S. patent application Ser. No. 10/797,490 filed Mar. 10, 2004 entitled “Artificial Christmas Tree;” which is a continuation-in-part of U.S. patent application Ser. No. 10/756,224 filed Jan. 13, 2004 entitled “Artificial Flower;” which is a continuation-in-party of U.S. Pat. No. 6,830,733 issued Dec. 14, 2004 entitled “Artificial Flower.” The contents of these applications and patent are expressly incorporated herein by reference.

### FIELD OF THE INVENTION

The present invention relates to stuffed toy fragrance devices, and more particularly to a powered fragrance dispenser that is adapted to be powered by battery pack.

### BACKGROUND OF THE INVENTION

Stuff Toys are well known and are sold and used throughout the world. Generally, stuffed toys are a novelty gift whose purpose is that of decoration and show of affection to those receiving them; lending themselves to the emotional betterment of the individual. There are a number of drawbacks to conventional stuffed toys. The novelty of receiving a stuff toy is generally effective for a short time period, leaving the individual with an item which has no further purpose. Yet, the individual typically does not wish to discharge the stuffed toy due to emotional attachment. This often result is the toy being used for decoration in the home or other location and often being added to what may already be a collection of stuffed toys, yet serving no other purpose for the owner. In a time where our lives require convenience as well as emotional satisfaction in our home decor, the stuffed toys give rise to a need for greater purpose.

Therefore, there has been and continues to be a need for a stuffed toy which produces emotional betterment, yet serves a purpose in improving the decor and overall atmosphere of our lives.

### SUMMARY OF THE INVENTION

An exemplary embodiment of the present invention entails a stuffed toy comprised of an ornamental object on the stuffed toy detachably coupled to a hollow stem. A fan, fragrance source and power source is provided and is emitted through the stuffed toy's flower or similar decoration.

Further, the present invention entails a method of generating or directing a fragrance that is associated with a stuffed toy. The method entails locating a fragrance source in a portion of the stuffed toy detachably coupled to a hollow stem and emitting a fragrance from the fragrance source.

Further, the present invention entails a method of generating or directing a fragrance that is associated with artificial potpourri. The method entails aggregating a stuffed toy and a fragrance source in one or more of the plurality of stuffed toy portions to emit a fragrance from the artificial potpourri.

Other objects and advantages of the present invention will become apparent and obvious from a study of the following description and the accompanying drawings, which are merely illustrative of such invention.

### BRIEF DESCRIPTION OF THE DRAWINGS

FIG. 1 is a plan view of the stuffed toy fragrance dispenser of the present invention.

FIG. 2 is a fragmentary side view of the fragrance dispenser illustrating the fan housing.

FIG. 3 is a view of the fragrance cartridge which is designed to attached to the fan housing.

FIG. 4 is a schematic illustration of the stuffed toy fragrance dispenser and which illustrates one embodiment of the same.

FIG. 5 is a view similar to FIG. 4, but illustrating a different ornamental object that forms a part of the fragrance dispenser.

FIG. 6 is a plan view of an alternate design for the stuffed toy where a fragrance source is disposed in a detachably coupled pouch with ornamental object.

FIG. 6A is a sectional view of pouch and ornamental object of FIG. 6.

### DESCRIPTION OF EXEMPLARY EMBODIMENT

With further reference to the drawings, the stuffed toy dispensing device of the present invention is shown therein and indicated generally by the numeral 10. As will be appreciated from the following description, the stuffed toy fragrance dispensing device 10 is designed to be utilized in a home, office or other location requiring decor. More particularly, the fragrance dispensing device 10 is designed to be powered from a battery pack or similar power source located within the stuffed toy or ornamental object. Once the stuffed toy fragrance dispensing device 10 has been activated by the battery pack or similar power source the power is used to drive a fan, which will in turn move air past a fragrance source where the air will mix with one or more fragrances to form an air-fragrance mixture which will ultimately be emitted from the device.

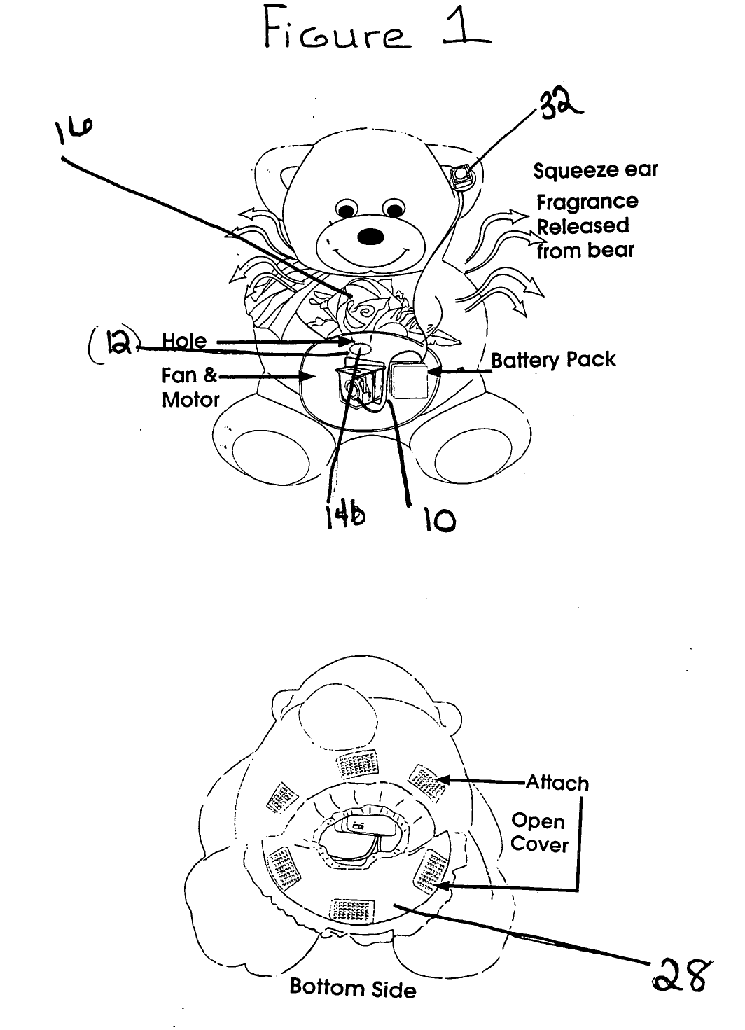

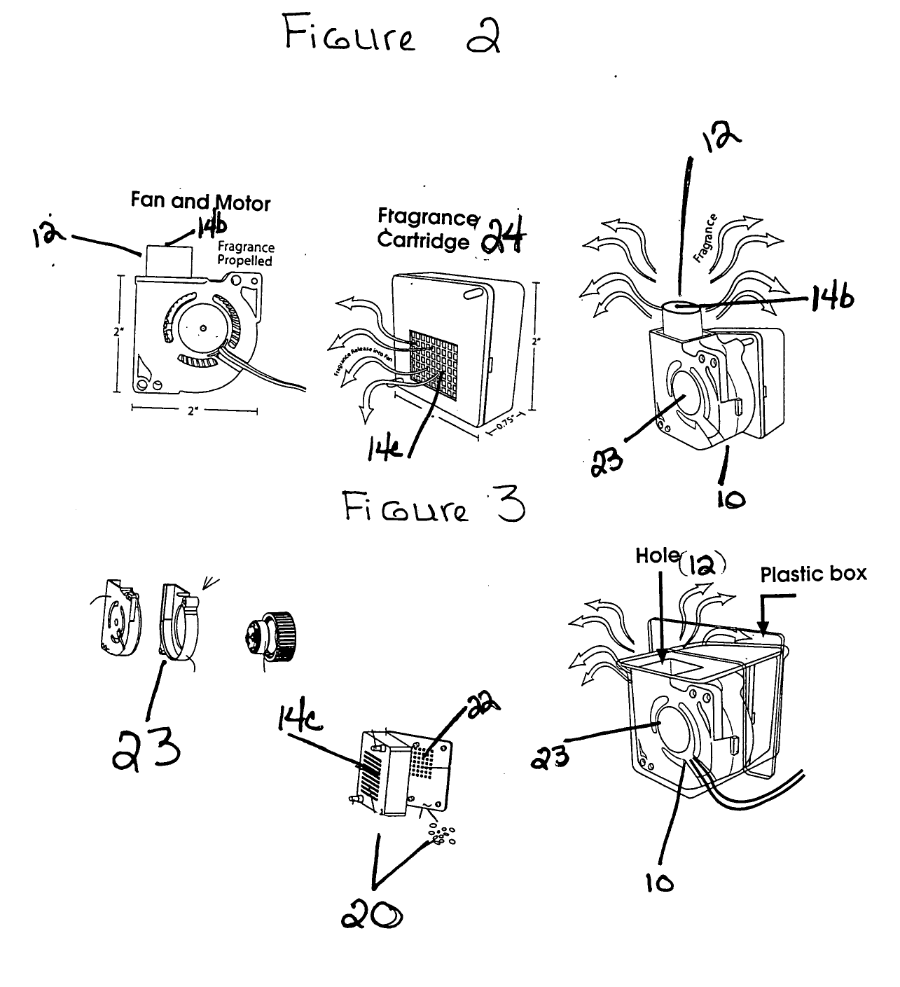

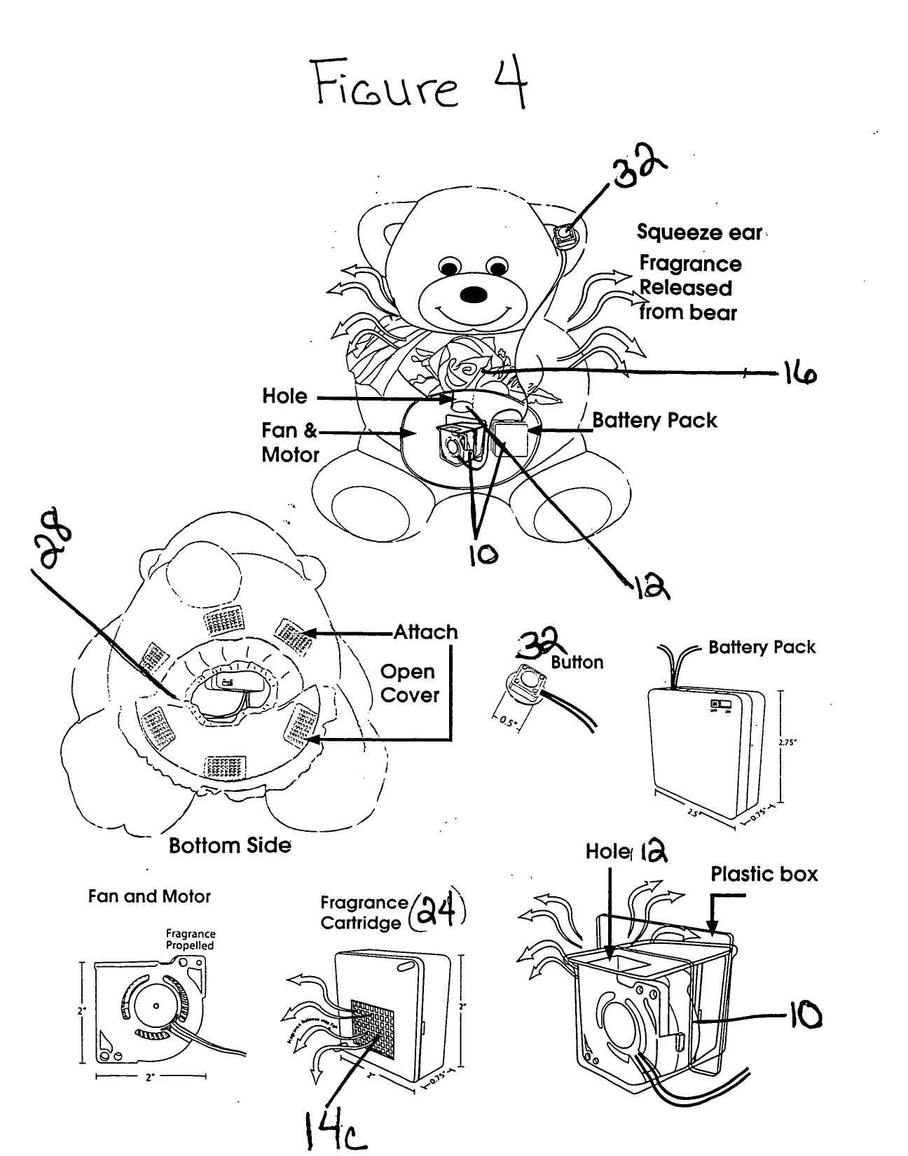

Turning now to the drawings, as shown in FIGS. 1-3, the fragrance dispensing device 10 includes a hollow stem indicated generally by the numeral 12 and ornamental object secured to or extending from a portion of the stem 12. The stuffed toy 10 can be made of various materials such as plush, plastics, or synthetic materials.

As seen in the drawings, the stem 12 is hollow. Stem 12 includes a surrounding wall structure 14 and a lower portion 14a that extends to an anchor end 18.

As will be described subsequently herein, the stem 12 is designed such that air and a fragrance can move through and, in at least one embodiment, is designed such that the fragrance can be emitted or dispersed from the upper portion 14b of the stem 12 into an environment where the ornamental object or other portion of the stuffed toy 16 resides. Openings 14c within the stem 12 permit air to enter the stem 12 and move upwardly through the hollow stem 12 towards the ornamental object 16.

It is appreciated that the ornamental object portion 16 may be secured or integrally formed with the stem 12 through various manufacturing and fabrication techniques. Ornamental object portion 16 may also assume various shapes and configurations.

The present invention entails associating a fragrance source, indicated generally by the numeral 20, with the stuffed toy 10 for dispersing fragrance into the environment. The fragrance source 20 can be of various conventional types. Further, the particular scent emitted by the fragrance source 20 may vary and may be selected to simulate or mimic the smell or scent of favored within the home, office, bedroom or other location. In addition, the size of the fragrance source 20 and/or the size of the air intake openings and/or outtake openings may affect the amount of fragrance dispersed into the environment.

Those skilled in the art will appreciate that the consumer may control any or all of these parameters and characteristics. For example, a consumer may select a particular scent by selecting one or more fragrance sources 20 for one or more stuffed toys 10. The consumer may also control the amount of dispersed fragrance by opening a slidable panel (not shown) to expose more openings in the container 22 or by varying the size of the intake openings. In addition, the amount or size of the fragrance source 20 can be varied. For example, in embodiments utilizing a solid fragrance source, the present invention may employ various sizes of such fragrance sources 20. In cases where a liquid or a semi-liquid fragrance is used, the quantity of the fragrance source 20 held within the artificial flower 10 can be varied.

In the embodiment illustrated in FIGS. 1-3, fragrance source 20 comprises of a fan 23 and scent cartridge 24 having a selected solid fragrance contained therein. The fragrance source 20 can be disposed in various locations about the stuffed toy 10. In the embodiment illustrated in FIG. 1, fragrance source 20 is disposed within the hollow stem 12.

Various mounting structures or mounting techniques can be utilized. For example, the fragrance source 20 can be set or held in the interior of the stuffed toy itself through an opening 28, thereby frictionally securing it to the stem 12. It will be appreciated that within the opening 28 the fragrance system may alternatively be secured to the interior of the stuffed toy 10 in some fashion (not shown) such as sewing, adhesive or by any other known securing means.

In the case of the embodiment shown in FIGS. 1-3, the fan 23 is a battery-powered fan that includes a main body held and supported within the upper portion 14b of the stem and including a fan blade, impeller, or propeller extending therefrom. A switch (not shown) may extend from the main body of the fan 23 outwardly through the stuffed toy 10 to a button 32 which can be used to externally activate the fragrance system. In addition, alternate embodiments of the present invention may also include power adapters so that fan 23 may plug into a wall outlet (not shown), vehicle power port, USB port or other source.

According to the present invention, fan 23 may provide a fixed airflow rate. Alternatively, fan 23 may provide a variable airflow rate. For example, fan 23 may be a multi-speed fan that enables a consumer to select the amount of fragrance dispersed into the environment by selecting a fan speed. In a preferred embodiment, a consumer selects a fan speed by pressing the button 32 located on the stuffed toy.

In the case of the design shown in FIG. 1, the fan 23 is disposed above the fragrance source 20. Therefore, the impeller or fan blade associated with the fan 30 is designed to induce or pull air from the scent cartridge 24 and through the openings 14c into the interior of the stem 12.

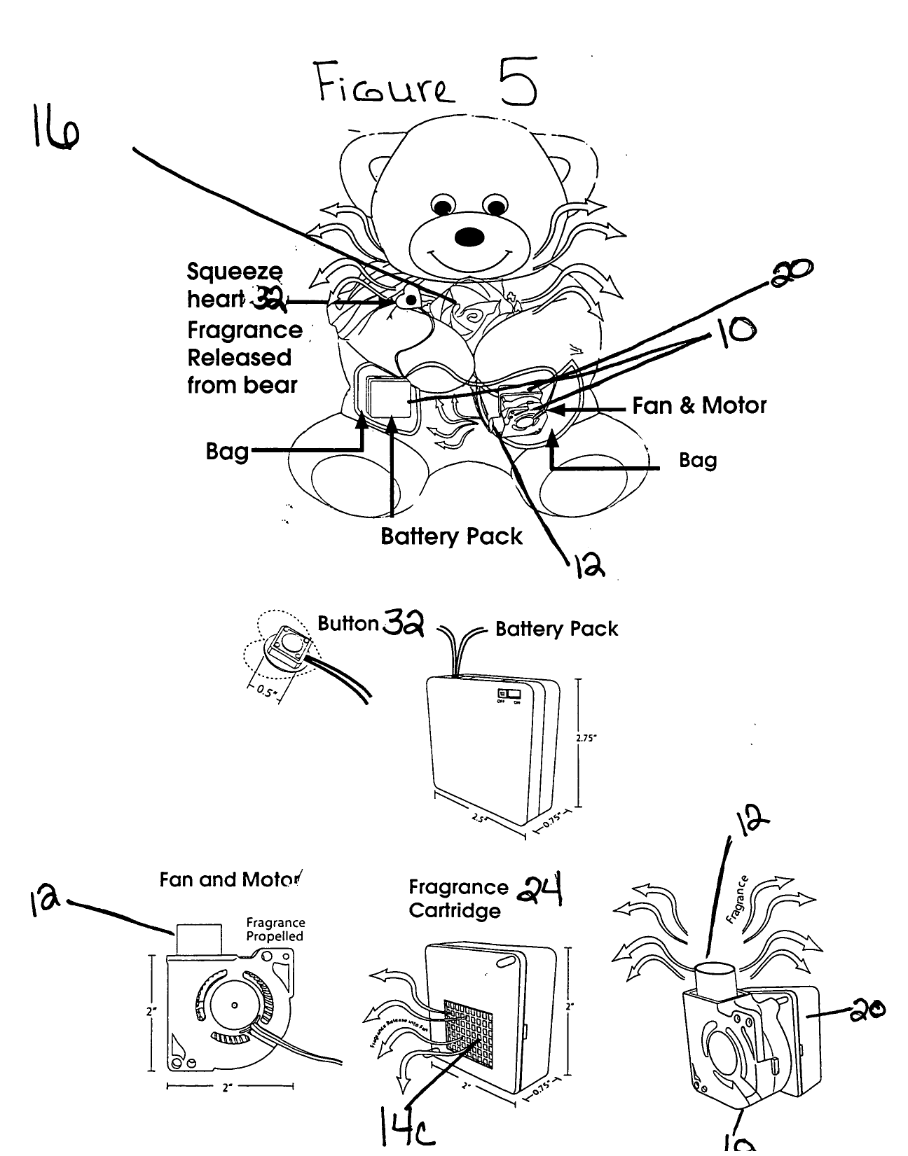

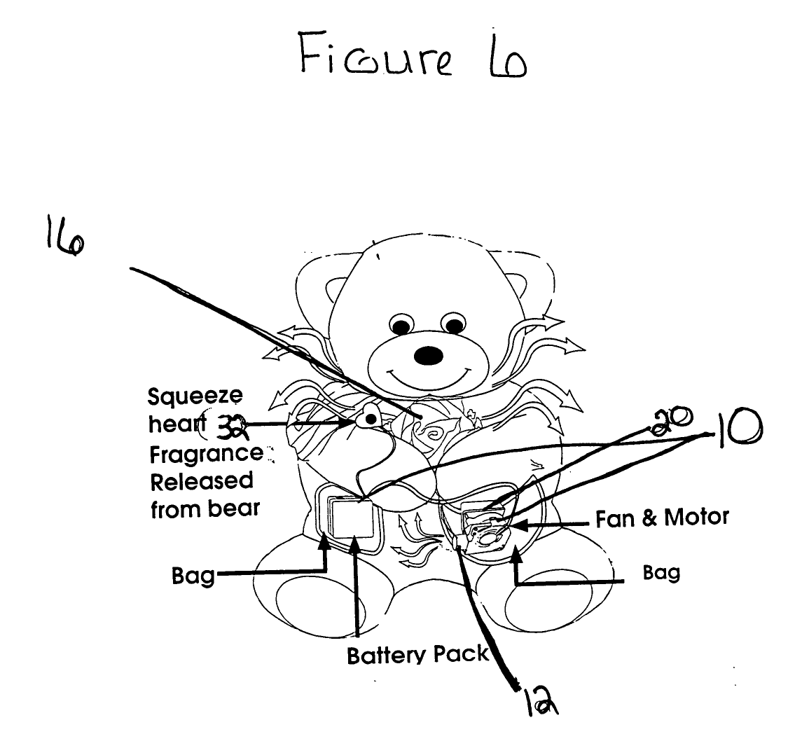

Accordingly, the fragrance from the scent cartridge 24 will be transferred to the passing air and ultimately will be dispersed out the upper portion 14b of the stem 12 to the ornamental object 16. Note also that the main body or frame of the fan 23 would be provided with openings that would enable air to be moved or pulled through the upper portion 14b of the stem 12, through the fan structure, and out of the upper end of the stem 12 to where the fragrance-laden air disperses into an area occupied by the ornamental object 16 as seen in FIGS. 1-5.

Turning to FIGS. 6 and 6A, an alternative embodiment for the stuffed toy 10 is shown therein. In this case, the fragrance source 20 is located within a detachably coupled pouch 30 located on the exterior of the stuffed toy 10. Similarly to the fragrance source 20 of the embodiment in FIG. 1-5, the fragrance will pass through the fragrance source 20 into the stem 12 and thereby release into the air.

It is appreciated that the present invention presents a stuffed toy which is designed to emit a pleasing fragrance or aroma. A fragrance source associated with the stuffed toy is designed to emit a fragrance that will be dispersed about the stuffed toy. Although the fragrance source can be disposed in various positions and locations with respect to the stuffed toy, in some embodiments, the fragrance source is disposed in the hollow stem that forms a part of an ornamental object of the stuffed toy. In other embodiments, the fragrance source is disposed in a portion of the stuffed toy. Moreover, the fragrance source may simply be designed or selected to be of the type that will slowly and over a period of time simply emit a fragrance that will move through or from the artificial flower.

The present invention may, of course, be carried out in other specific ways than those herein set forth without departing from the scope and the essential characteristics of the invention. The present embodiments are therefore to be construed in all aspects as illustrative and not restrictive and all changes coming within the meaning and equivalency range of the appended claims are intended to be embraced therein.

Various structures, systems and methods can be employed for dispersing the fragrance from the stuffed toy. These have been disclosed in the parent applications: U.S. patent application Ser. No. 10/164,818, filed Jun. 7, 2002 and entitled “Artificial Flower,” and U.S. patent application Ser. No. 10/756,224 filed Jan. 13, 2004 and entitled “Artificial Flower.” These disclosures are expressly incorporated herein by reference. In these applications, various artificial flowers have been disclosed and wherein the artificial flowers are provided with means for emitting a fragrance. Various types of fragrance delivery systems have been disclosed in connection with these artificial flowers. The same fragrance delivery systems are applicable to the stuffed toy fragrance dispensing system of the present invention and therefore details of such will not be dealt with herein. However, as discussed in the three patent applications incorporated herein, the fragrance source may assume various forms including a liquid, a gas or a solid. Further, as discussed in the case of the Artificial Flower, a source of heat may be utilized in conjunction with the fragrance source to facilitate the emission of the fragrance from the fragrance source.

In addition, as disclosed with respect to the Artificial Flower, the fragrance source may be positioned at various locations with respect to the Christmas stuffed toy. The fragrance source may be contained in a container which may be disposed adjacent the stuffed toy and provided with communication or conduit means for channeling the fragrance from the fragrance source 20 to the stuffed toy 10. In addition, the fragrance source can be stationed in the stuffed toy itself, again at various locations, in an opening located underneath 28 for example.

As noted above, there are various ways to direct the air-fragrance mixture through the stuffed toy 10. One particular way is similar to that shown in FIGS. 1 and 6 with respect to the stuffed toy 10.

The present invention may, of course, be carried out in other specific ways than those herein set forth without departing from the scope and the essential characteristics of the invention. The present embodiments are therefore to be construed in all aspects as illustrative and not restrictive and all changes coming within the meaning and equivalency range of the appended claims are intended to be embraced therein.

1. A stuffed toy comprising of a fragrance source for emitting a fragrance.

2. The stuffed toy of claim 1 further including diffuser to move air across the fragrance source.

3. The stuffed toy of claim 2 wherein the diffuser comprises a fan.

4. The stuffed toy of claim 2 wherein the diffuser comprises a heat source.

5. The stuffed toy of claim 2 where at least a portion of the diffuser is disposed within the stuffed toy.

6. The stuffed toy of claim 5 wherein the diffuser is disposed within a pouch or ornamental object attached to the stuffed toy.

7. The stuffed toy of claim 1 wherein the fragrance source includes a container having a fragrance contained therein.

8. The stuffed toy of claim 1 wherein the fragrance source is disposed within the stuffed toy itself.

9. A method of generating a fragrance for a stuffed toy comprising: associating a fragrance source with the stuffed toy and emitting a fragrance from the source such that the fragrance imparts a scent about the stuffed toy.

10. The method of claim 9 comprising directing air across the fragrance source to emit a fragrance from the stuffed toy.

11. The method of claim 10 wherein directing air across the fragrance source comprises locating a fan within a portion of the stuffed toy and directing air from the fan across the fragrance source.

12. The method of claim 11 comprising locating the fan within a portion of the stuffed toy.

13. The method of clam 10 further comprising locating a heater within at least a portion of the stuffed toy and directing air heated by the heating source across the fragrance source.

14. The method of clam 10 wherein associating a fragrance source with the stuffed toy comprises locating a fragrance container within or adjacent the stuffed toy, said fragrance container having a fragrance contained therein.

15. The method of claim 14 further comprising replacing the fragrance source by refilling the fragrance container with fragrance.
